(19) United States
(12) Patent Application Publication
(10) Pub. No.: US 2018/0008567 A1
(43) Pub. Date: Jan. 11, 2018

(54) COMPOSITIONS COMPRISING 15-HEPE AND METHODS OF USING THE SAME

(71) Applicant: Afimmune Limited, Dublin (IE)

(72) Inventors: John Climax, Dublin (IE); David Coughlan, Dublin (IE); Mehar Manku, Birmingham (GB)

(21) Appl. No.: 15/542,668

(22) PCT Filed: Jan. 15, 2016
(86) PCT No.: PCT/IB2016/000202

§ 371 Date: Jul. 11, 2017

Related U.S. Application Data
(60) Provisional application No. 62/104,472, filed on Jan. 16, 2015.

(51) Int. Cl. A61K 31/202; A61K 9/48; A61K 9/00

(52) U.S. Cl. CPC A61K 31/202; A61K 9/0053; A61K 9/48

(57) ABSTRACT

The present disclosure relates to the compositions, formulations and methods of treating or preventing diseases mediated by peroxisome proliferator-activated receptors (PPARs) by administration of 15-HEPE.

### PRIORITY

This application is a 371 U.S. National Stage application of International PCT Application No. PCT/IB2016/000202, filed Jan. 15, 2016, which claims priority from U.S. Provisional Application Ser. No. 62/104,472 filed on Jan. 16, 2015, the entirety of which is incorporated by reference herein.

### BACKGROUND

Peroxisome proliferator-activated receptors (PPARs) are ligand-activated transcription factors of nuclear hormone receptor superfamily comprising of the following three subtypes: PPARα, PPARγ, and PPARβ/δ. Activation of PPAR-α reduces triglyceride level and is involved in regulation of energy homeostasis. Activation of PPAR-γ causes insulin sensitization and enhances glucose metabolism, whereas activation of PPAR-β/δ enhances fatty acids metabolism. Thus, PPAR family of nuclear receptors plays a major regulatory role in energy homeostasis and metabolic function.

### SUMMARY OF THE INVENTION

In various embodiments, the invention provides compositions, formulations and methods of treating diseases and disorders mediated by peroxisome proliferator-activated receptors (PPARs). In various embodiments, such diseases and disorders include impaired insulin sensitivity, psoriasis, cancer (e.g. melanoma), neurodegenerative disorders (e.g. Huntington's disease), inflammatory diseases, adipocyte differentiation, fertility or reproduction issues, pain, obesity, and their sequelae by administration of a pharmaceutical composition comprising 15-hydroxy eicosapentaenoic acid (hereinafter “15-HEPE”) in a subject in need thereof.

### BRIEF DESCRIPTION OF THE FIGURES

FIG. 1 shows activation of human PPARs by 15-HEPE ethyl ester at 100,000 nm concentration compared to DMSO (0.10%), EPA ethyl ester (100,000 nm) and GW590735 (PPAR-α), GW0742 (PPAR-δ) or rosiglitazone (PPAR-γ).

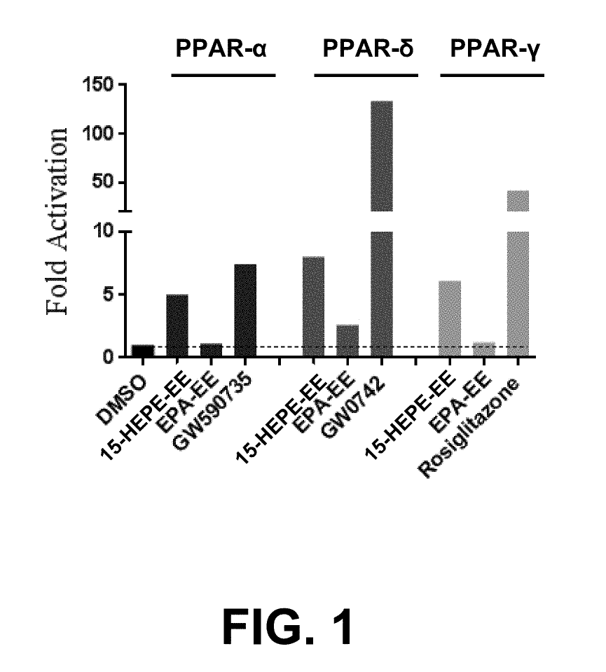

FIG. 2 shows activation of human PPARs by 15-HEPE ethyl ester at 11,111 nm concentration compared to DMSO (0.10%) and EPA ethyl ester (11,111 nm).

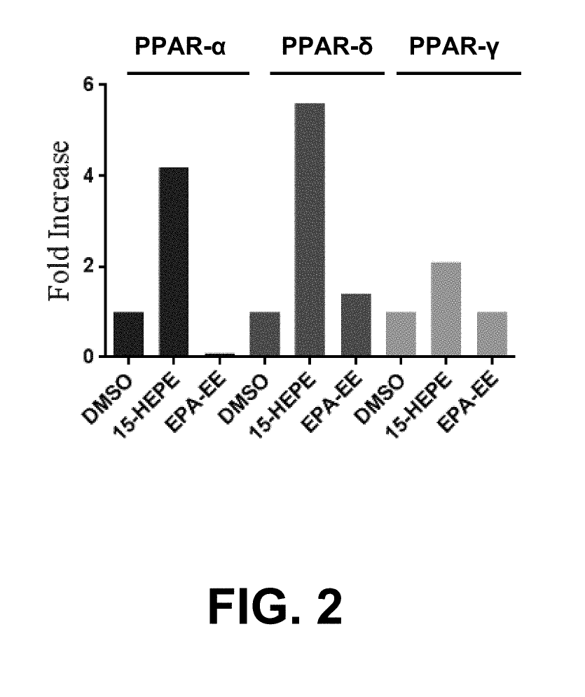

FIG. 3 shows activation of human PPARs by 15-HEPE ethyl ester at 33,333 nm concentration compared to DMSO (0.10%) and EPA ethyl ester (33,333 nm).

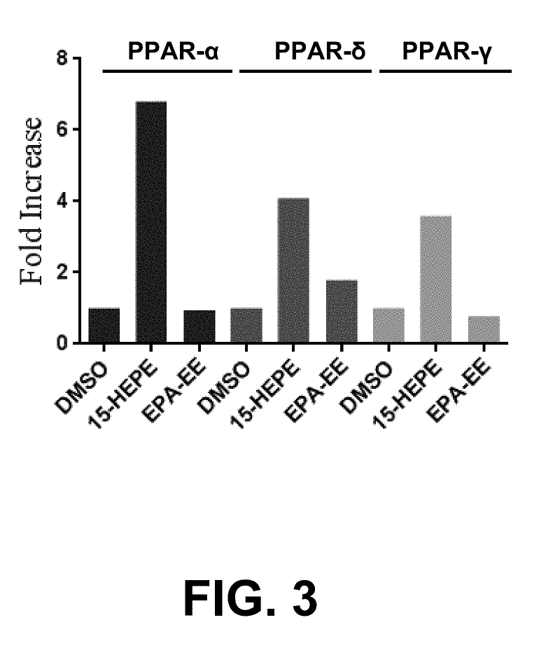

FIG. 4 shows activation of human PPARs by 15-HEPE ethyl ester at 100,000 nm concentration compared to DMSO (0.10%) and EPA ethyl ester (100,000 nm).

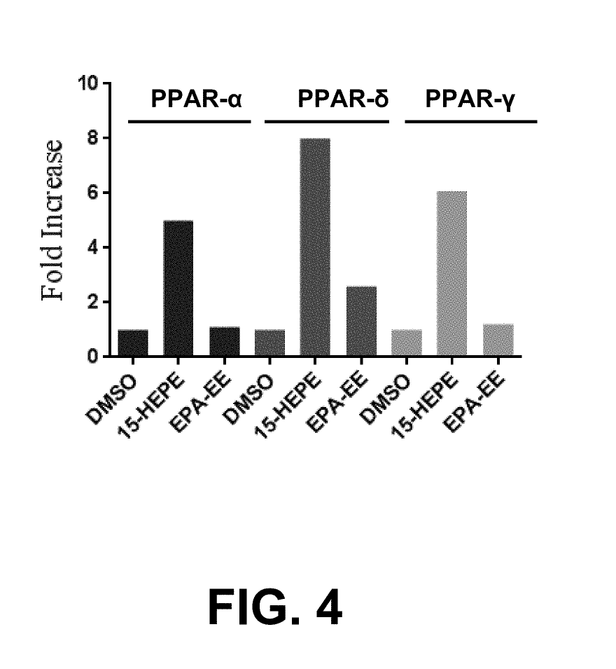

FIG. 5 shows activation of rat PPARs by 15-HEPE ethyl ester at 33,333 nm concentration compared to DMSO (0.10%) and EPA ethyl ester (33,333 nm).

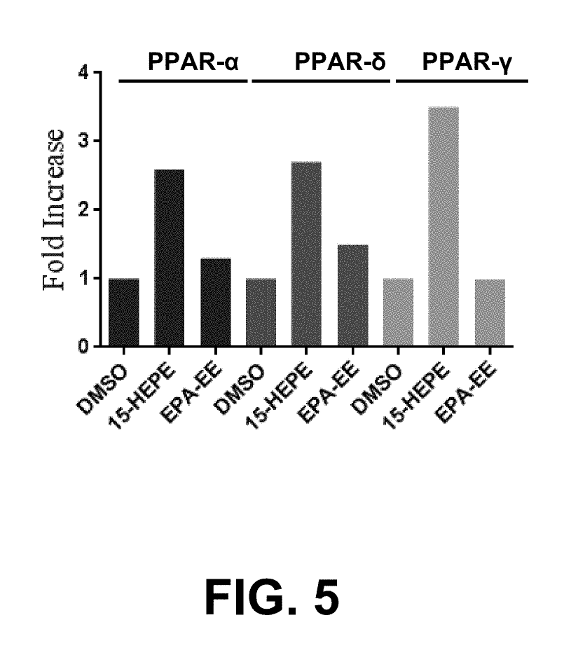

### DETAILED DESCRIPTION

The present invention relates to compositions, formulations and methods of treating diseases and disorders mediated by peroxisome proliferator-activated receptors (PPARs) by administration of a pharmaceutical composition comprising 15-HEPE in a subject in need thereof.

As used herein, “15-HEPE” is 15-Hydroxy-eicosa-5,8,11,13,17-pentaenoic acid. 15-HEPE, also occasionally referred to as 15-OHEPA, can be synthesized from eicosapentaenoic acid (“EPA,” eicosa-5,8,11,14,17-pentaenoic acid or 20:5n-3), an omega-3 fatty acid according to methods known in the art. As used herein, the term “15-HEPE” refers to 15-HEPE in its free acid form (e.g, 15-hydroxy-eicosa-5,8,11,13,17-pentaenoic acid) and/or a pharmaceutically acceptable ester, conjugate or salt thereof, or mixtures of any of the foregoing. A derivative of 15-HEPE may be used instead, though this does not include any derivative compound missing the hydroxy group of 15-HEPE. In some embodiments, the 15-HEPE is used in the free acid form. Alternatively, pharmaceutically acceptable esters or salts of 15-HEPE are used in the invention. In some embodiments, the 15-HEPE is in the form of a C₁₋₄ alkyl ester such as methyl ester or ethyl ester form.

Accordingly, in one aspect of the present invention, a method of treating a PPAR-mediated disease or disorder in a subject is provided, comprising administering to the subject a therapeutically effective amount of a composition comprising 15-HEPE. In various embodiments, the PPAR-mediated disease or disorder is selected from: impaired insulin sensitivity, psoriasis, cancer (e.g. melanoma), neurodegenerative disorders (e.g. Huntington's disease) and inflammatory diseases.

The present invention provides a use of 15-HEPE, or a composition comprising 15-HEPE, in the manufacture of a medicament for treating a PPAR-mediated disease or disorder, for example impaired insulin sensitivity, psoriasis, cancer (e.g. melanoma), neurodegenerative disorders (e.g. Huntington's disease) and inflammatory diseases.

In another aspect, the present invention provides a pharmaceutical composition comprising a therapeutically effective amount of 15-HEPE. The 15-HEPE may be the sole significant active ingredient in that composition and in the methods and uses as stated herein. The 15-HEPE may be the sole active ingredient. Alternatively, the 15-HEPE may be combined for co-formulation or co-administration with other agents for treating a PPAR-mediated disease or disorder. If an additional active agent is to be used, the 15-HEPE can be co-formulated as a single dosage unit or can be formulated as two to a plurality of dosage units for coordinated, combination or concomitant administration.

The invention also provides formulations of 15-HEPE and formulations comprising 15-HEPE and methods of using these formulations for treating a PPAR-mediated disease or disorder.

15-HEPE is a chiral molecule and may be used in the (S)- or (R)-enantiomeric form, or as a racemic mixture. Used herein, “15-HEPE” includes all such forms, with no limitation as to stereospecifcity. In another embodiment, the 15-HEPE comprises the (S) form: 15(S)-Hydroxy-(5Z,8Z,11Z,13E,17Z)-eicosapentaenoic acid. In some embodiments, the 15-HEPE may be used in the form of the ethyl ester. In other embodiments the 15-HEPE may be used as the free acid.

The present invention further provides an pharmaceutical composition for oral delivery, comprising 15-HEPE. That composition may comprise a pharmaceutically acceptable excipient. The 15-HEPE may be in any form as discussed herein. The 15-HEPE may be present from about 50 mg to about 3000 mg.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from the following detailed description.

### Pharmaceutical Compositions

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” In this manner, slight variations from a stated value can be used to achieve substantially the same results as the stated value. Also, the disclosure of ranges is intended as a continuous range including every value between the minimum and maximum values recited as well as any ranges that can be formed by such values. Also disclosed herein are any and all ratios (and ranges of any such ratios) that can be formed by dividing a recited numeric value into any other recited numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

### 15-Hydroxy Eicosapentaenoic Acid

In one embodiment, compositions of the invention comprise 15-HEPE as an active ingredient. 15-HEPE is the abbreviation for 15-Hydroxy eicosapentaenoic acid, a metabolite of eicosapentaenoic acid (EPA) that can be synthesized via methods known in the art, such as exposure of eicospentaenoic acid to the enzyme 15-lipoxygenase . As used herein, the term “15-HEPE” refers to 15-HEPE in its free acid form (e.g., 15-Hydroxy eicosapentaenoic acid) and/or a pharmaceutically acceptable ester, conjugate or salt thereof, or mixtures of any of the foregoing. A derviative of 15-HEPE may be used instead, though this does not include any derivative compound missing the hydroxy group of 15-HEPE. The term “pharmaceutically acceptable” in the present context means that the substance in question does not produce unacceptable toxicity to the subject or interaction with other components of the composition.

In one embodiment, the 15-HEPE is in the form of an ester (also referred to herein as E-15-HEPE or ethyl-15-HEPE). In another embodiment, the 15-HEPE comprises a C₁-C₅ alkyl ester of 15-HEPE. In another embodiment, the 15-HEPE comprises 15-HEPE methyl ester, 15-HEPE propyl ester, or 15-HEPE butyl ester. In still another embodiment, the 15-HEPE comprises the optically active 15(S)-Hydroxy-(5Z,8Z,11Z,13E,17Z)-eicosapentaenoic acid. This isomer may be used in any of the forms discussed above.

In another embodiment, the 15-HEPE comprises lithium 15-HEPE, mono, di- or triglyceride 15-HEPE or any other ester or salt of 15-HEPE, or the free acid form of 15-HEPE.

In various embodiments, the invention provides pharmaceutical compositions, for example orally deliverable compositions, comprising 15-HEPE. In one embodiment, the compositions comprise a therapeutically effective amount of 15-HEPE. In one embodiment, the pharmaceutical composition comprises about to about 99%, about 1% to about 95%, about 5% to about 90% by weight of 15-HEPE.

In one embodiment, the pharmaceutical composition comprises about at least about 70%, at least about 80% or at least about 90%, by weight, of 15-HEPE. In one embodiment, the pharmaceutical composition comprises at least about 50%, at least about 60%, at least about 70%, at least about 80% or at least about 90%, by weight of 15-HEPE.

In another embodiment, 15-HEPE is present in a composition of the invention in an amount of about 1 mg to about 10,000 mg, 25 mg to about 7500 mg, about 25 mg to about 5000 mg, about 50 mg to about 5000 mg, about 50 mg to about 3000 mg, about 75 mg to about 2500 mg, or about 100 mg to about 1000 mg, for example about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, or about 2500 mg.

In one embodiment, 15-HEPE present in a composition of the invention comprises at least 90% by weight 15-HEPE (as the term “15-HEPE” is defined and exemplified herein). 15-HEPE compositions can comprise even higher purity 15-HEPE, for example at least 95% by weight 15-HEPE or at least 97% by weight 15-HEPE, wherein the 15-HEPE is any form of 15-HEPE as set forth herein. The purity of 15-HEPE can further be defined (e.g. impurity profile) by any of the descriptions of 15-HEPE provided herein.

Above are discussed the amounts of the 15-HEPE in the pharmaceutical composition and their purity. The nature of the essential fatty acids and their synthesis is such that the 15-HEPE composition may include moieties from other essential fatty acids in the essential fatty acid metabolic cascade.

In one embodiment, a composition of the invention contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight of other omega-3 fatty acids including alpha linolenic acid, stearidonic acid, docosahexaenoic acid (DHA) or derivatives thereof. In other embodiments there is substantially no, or no such other omega-3 fatty acids present.

In another embodiment, 15-HEPE represents at least about 60%, at least about 70%, at least about 80%, at least about 90%, at least about 95%, at least about 97%, at least about 98%, at least about 99%, or 100%, by weight, of all fatty acids present in a composition of the invention.

There may be present some residual eicosapentaenoic acid from the synthesis of the 15-HEPE. There may be not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight EPA. Alternatively, there is substantially no, or no, EPA in a form which has not been modified to the hydroxyl-form.

### Additional Active Agents

In one embodiment, the pharmaceutical composition further comprises one or more additional active agent(s). In one embodiment, the pharmaceutical composition comprises an amount of the additional active agent that is less than the generally recognized therapeutically effective amount for that agent. In one embodiment, the pharmaceutical composition comprises an amount of the additional active agent that is equal to or greater than the generally recognized therapeutically effective amount for that agent.

EPA itself has beneficial properties in treating FLD and it is possible to combine the 15-HEPE with EPA in an alternative embodiment.

In one embodiment, 15-HEPE and one or more active agent(s) are present in a composition of the invention, or are co-administered in a weight ratio of 15-HEPE: additional agent of about 1:1000 to about 1000:1, about 1:500 to about 500:1, about 1:100 to about 100:1, about 1:50 to about 50:1, about 1:25 to about 25:1, about 1:10 to about 10:1, about 1:5 to about 5:1, about 1:4 to about 4:1 about 1:3 to about 3:1, about 1:2 to about 2:1 or about 1:1.

### Dosage Forms

A composition for use in accordance with the disclosure can be formulated as one or more dosage units. The terms “dose unit” and “dosage unit” herein refer to a portion of a pharmaceutical composition that contains an amount of a therapeutic agent suitable for a single administration to provide a therapeutic effect. Such dosage units may be administered one to a plurality (i.e. 1 to about 10, 1 to 8, 1 to 6, 1 to 4 or 1 to 2) of times per day, or as many times as needed to elicit a therapeutic response.

In some embodiments, compositions of the invention are in the form of orally deliverable dosage forms or units. Non-limiting examples of suitable dosage forms include tablets (e.g. suspension tablets, bite suspension tablets, rapid dispersion tablets, chewable tablets, etc), caplets, capsules (e.g. a soft or a hard gelatin capsule or HPMC capsule), lozenges, sachets, cachets, troches, pellets, suspension, elixirs, syrups or any other solid dosage form reasonably adapted for oral administration. The terms “oral delivery” and “oral administration” herein include any form of delivery wherein the agent or composition is placed in the mouth of the subject under treatment, whether swallowed or not. This therefore includes buccal and sublingual administration, as well as esophagael administration.

Alternatively, compositions of the invention can also be formulated for rectal, topical, or parenteral (e.g. subcutaneous, intramuscular, intravenous and intradermal or infusion) delivery.

In discussing the amount of 15-HEPE in a composition of the invention, this may be split over several dosage forms. There is a limit as to the size for oral administration. If a subject is to be administered 1 to 4 g 15-HEPE a day, this may be by up to 4 capsules, each providing 1 g of 15-HEPE.

Compositions of the invention can be in the form of liquid dosage forms or dose units to be imbibed directly or they can be mixed with food or beverage prior to ingestion. Non-limiting examples of suitable liquid dosage forms include solutions, suspensions, elixirs, syrups, liquid aerosol formulations, and the like.

In another embodiment, compositions of the invention comprise one or more pharmaceutically acceptable excipients. The term “pharmaceutically acceptable excipient” herein means any substance, not itself a therapeutic agent, used as a carrier or vehicle for delivery of a therapeutic agent to a subject or added to a pharmaceutical composition to improve its handling or storage properties or to permit or facilitate formation of a unit dose of the composition, and that does not produce unacceptable toxicity or interaction with other components in the composition. By way of example only, a pharmaceutical composition according to the present disclosure may comprise one or more of: antioxidants, surfactants, preservatives, flavouring agents, co-solvents, viscosity aids, suspension aids, and lipophilic phases.

In one embodiment, the pharmaceutical composition comprises one or more antioxidants such as ascorbic acid, palmitic acid, ascorbyl palmitate, α-tocopherol, idebenone, ubiquinone, ferulic acid, coenzyme Q10, lycopene, green tea, catechins, epigallocatechin 3-gallate (EGCG), green tea polyphenols (GTP), silymarin, coffeeberry, resveratrol, grape seed, pomegranate extracts, genisten, pycnogenol, niacinamide, and the like. In one embodiment, the pharmaceutical composition comprises about 0.01 wt. % to about 2 wt. % of an antioxidant, for example about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.11 wt. %, about 0.12 wt. %, about 0.13 wt. %, about about 0.15 wt. %, about 0.16 wt. %, about 0.17 wt. %, about 0.18 wt. %, about 0.19 wt. %, about 0.2 wt. % about 0.21 wt. %, about 0.22 wt. %, about 0.23 wt. %, about 0.24 wt. %, about 0.25 wt. %, about 0.26 wt. %, about 0.27 wt. %, about 0.28 wt. %, about 0.29 wt. %, about 0.3 wt. %, about 0.31 wt. %, about 0.32 wt. %, about 0.33 wt. %, about 0.34 wt. %, about 0.35 wt. %, about 0.36 wt. %, about 0.37 wt. %, about 0.38 wt. %, about 0.39 wt. %, about 0.4 wt. %, about 0.41 wt. %, about 0.42 wt. %, about 0.43 wt. %, about 0.44 wt. %, about 0.45 wt. %, about 0.46 wt. %, about 0.47 wt. %, about 0.48 wt. %, about 0.49 wt. %, about 0.5 wt. %, about 0.51 wt. %, about 0.52 wt. %, about 0.53 wt. %, about 0.54 wt. %, about 0.55 wt. %, about 0.56 wt. %, about 0.57 wt. %, about 0.58 wt. %, about 0.59 wt. %, about 0.6 wt. %, about 0.61 wt. %, about 0.62 wt. %, about 0.63 wt. %, about about 0.65 wt. %, about 0.66 wt. %, about 0.67 wt. %, about 0.68 wt. %, about 0.69 wt. %, about 0.7 wt. %, about 0.71 wt. %, about 0.72 wt. %, about 0.73 wt. %, about 0.74 wt. %, about 0.75 wt. %, about 0.76 wt. %, about 0.77 wt. %, about 0.78 wt. %, about 0.79 wt. %, about 0.8 wt. %, about 0.81 wt. %, about 0.82 wt. %, about 0.83 wt. %, about 0.84 wt. %, about 0.85 wt. %, about 0.86 wt. %, about 0.87 wt. %, about 0.88 wt. %, about 0.89 wt. %, about 0.9 wt. %, about 0.91 wt. %, about 0.92 wt. %, about 0.93 wt. %, about 0.94 wt. %, about 0.95 wt. %, about 0.96 wt. %, about 0.97 wt. %, about 0.98 wt. %, about 0.99 wt. %, about 1 wt. %, about 1.1 wt. %, about 1.2 wt. % about 1.3 wt. % about 1.4 wt. % about 1.5 wt. % about 1.6 wt. %, about 1.7 wt. %, about 1.8 wt. %, about 1.9 wt. %, or about 2 wt. % of the one or more antioxidant.

### Therapeutic Methods

The compositions and formulations disclosed herein may be used in the treatment of a PPAR-mediated disease or disorder. In one embodiment the PPAR-mediated disease or disorder is selected from impaired insulin sensitivity, psoriasis, cancer (e.g. melanoma), firbrosis, neurodegenerative disorders (e.g. Huntington's disease), inflammatory diseases, adipocyte differentiation, fertility or reproduction issues, pain, obesity, and their sequelae.

In one embodiment, the present disclosure provides a method of treating and/or preventing impaired insulin sensitivity in a subject, the method comprising administering to the subject an effective amount of a composition comprising 15-HEPE. In some embodiments, the method further comprises determining that the subject is sensitive to insulin and/or is at risk of developing insulin sensitivity before administering the composition comprising 15-HEPE.

In one embodiment, the present disclosure provides a method of treating and/or preventing psoriasis in a subject, the method comprising administering to the subject an effective amount of a composition comprising 15-HEPE. In some embodiments, the method further comprises determining that the subject has psoriasis and/or is at risk of developing psoriasis before administering the composition comprising 15-HEPE.

In one embodiment, the present disclosure provides a method of treating and/or preventing cancer in a subject, the method comprising administering to the subject an effective amount of a composition comprising 15-HEPE. In some embodiments, the method further comprises determining that the subject has cancer and/or is at risk of developing cancer before administering the composition comprising 15-HEPE. In some embodiments, the cancer is a skin cancer. In some embodiments, the skin cancer is melanoma.

In one embodiment, the present disclosure provides a method of treating and/or preventing a neurodegenerative disorder in a subject, the method comprising administering to the subject an effective amount of a composition comprising 15-HEPE. In some embodiments, the method further comprises determining that the subject has a neurodegenerative disorder and/or is at risk of developing a neurodegenerative disorder before administering the composition comprising 15-HEPE. In some embodiments, the neurodegenerative disorder is Huntington's disease.

In one embodiment, the present disclosure provides a method of treating and/or preventing an inflammatory disease in a subject, the method comprising administering to the subject an effective amount of a composition comprising 15-HEPE. In some embodiments, the method further comprises determining that the subject has an inflammatory disease and/or is at risk of developing an inflammatory disease before administering the composition comprising 15-HEPE.

In one embodiment, the present disclosure provides a method of treating and/or preventing an adipocyte differentiation disorder in a subject, the method comprising administering to the subject an effective amount of a composition comprising 15-HEPE. In some embodiments, the method further comprises determining that the subject has an adipocyte differentiation disorder and/or is at risk of developing an adipocyte differentiation disorder before administering the composition comprising 15-HEPE.

In one embodiment, the present disclosure provides a method of treating and/or preventing fertility or reproduction issues in a subject, the method comprising administering to the subject an effective amount of a composition comprising 15-HEPE. In some embodiments, the method further comprises determining that the subject has fertility or reproduction issues and/or is at risk of developing fertility or reproduction issues before administering the composition comprising 15-HEPE.

In one embodiment, the present disclosure provides a method of treating and/or preventing pain in a subject, the method comprising administering to the subject an effective amount of a composition comprising 15-HEPE. In some embodiments, the method further comprises determining that the subject has pain and/or is at risk of developing pain before administering the composition comprising 15-HEPE.

In one embodiment, the present disclosure provides a method of treating and/or preventing obesity in a subject, the method comprising administering to the subject an effective amount of a composition comprising 15-HEPE. In some embodiments, the method further comprises determining that the subject is obese and/or is at risk of becoming obese before administering the composition comprising 15-HEPE.

As used herein, “treating” or “treatment” of a disease, disorder, or condition includes at least partially: (1) preventing the disease, disorder, or condition, i.e. causing the clinical symptoms of the disease, disorder, or condition not to develop in a mammal that is exposed to or predisposed to the disease, disorder, or condition but does not yet experience or display symptoms of the disease, disorder, or condition; (2) inhibiting the disease, disorder, or condition, i.e., arresting or reducing the development of the disease, disorder, or condition or its clinical symptoms; or (3) relieving the disease, disorder, or condition, i.e., causing regression of the disease, disorder, or condition or its clinical symptoms. The term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

An “effective amount,” as used herein, refers to the amount of an active composition that is required to confer a therapeutic effect on the subject. A “therapeutically effective amount,” as used herein, refers to a sufficient amount of an agent or a compound being administered which will relieve to some extent one or more of the symptoms of the disease, disorder, or condition being treated. In some embodiments, the result is a reduction and/or alleviation of the signs, symptoms, or causes of a disease, or any other desired alteration of a biological system. For example, in some embodiments, an “effective amount” for therapeutic uses is the amount of the composition including a compound as disclosed herein required to provide a clinically significant decrease in disease symptoms without undue adverse side effects. In some embodiments, an appropriate “effective amount” in any individual case is determined using techniques, such as a dose escalation study. The term “therapeutically effective amount” includes, for example, a prophylactically effective amount. In other embodiments, an “effective amount” of a compound disclosed herein, such as a compound of Formula (A) or Formula (I), is an amount effective to achieve a desired pharmacologic effect or therapeutic improvement without undue adverse side effects. In other embodiments, it is understood that “an effect amount” or “a therapeutically effective amount” varies from subject to subject, due to variation in metabolism, age, weight, general condition of the subject, the condition being treated, the severity of the condition being treated, and the judgment of the prescribing physician. The term “pharmaceutically acceptable” in the present context means that the substance in question does not produce unacceptable toxicity to the subject or interaction with other components of the composition.

Without further description, it is believed that one of ordinary skill in the art may, using the preceding description and the following illustrative examples, make and utilize the agents of the present disclosure and practice the claimed methods. The following working examples are provided to facilitate the practice of the present disclosure, and are not to be construed as limiting in any way the remainder of the disclosure.

### EXAMPLES

### Example 1

### PPAR Agonist Activities

This example demonstrates that 15-HEPE has moderate agonism activity against human PPARα, PPARδ, and PPARγ at low to higher doses in the dose response curve, and mild to moderate agonism activity against rat PPARα, PPARδ, and PPARγ at medium to higher doses in the dose response curve.

### 1.1 Study Design

This study utilized proprietary reporter cells expressing a hybrid receptor comprising the N-terminal Gal4 DNA binding domain fused to the ligand binding domain of the specific human nuclear receptor (hPPARα, hPPARδ, and hPPARγ) and rat nuclear receptor (rPPARα, rPPARδ, and rPPARγ). The reporter vectors used in this Example comprise the firefly luciferase gene functionally linked to the Gal4 upstream activation sequence.

### 1.2 Methods

Nuclear Receptor assay was performed by dispensing 100 μL of a suspension of Reporter Cells in Cell Recovery Medium (CRM) containing 10% charcoal stripped FBS into each well of a white 96-well plate. Test compounds were diluted using compound screening medium (CSM) containing 10% charcoal stripped FBS to generate “2×-concentration” treatment media. Immediately after this dilution step, 100 μL of each diluted 2×-concentration treatment medium was dispensed (in triplicate) into the Reporter Cell-containing assay wells.

After incubation at 37° C. for 24 hours, the treatment media was discarded. 100 μL of Luciferase Detection Reagent substrate was added to each well and RLUs were quantified from each well to determine nuclear receptor activities.

### 1.3 Results

Agonist assay results are shown in Table 1 below for 15-HEPE (ethyl ester, (S)-enantiomer). For comparison, agonist assay results for 15-HEPE's corresponding 15-lipoxygenase precursor, EPA ethyl ester, are also shown. Assay results for vehicle (DMSO) only were used to normalize the data (activity=1.0).

TABLE 1
Agonist Assay Results vs. Vehicle

| | | Fold−Activation (vs. 0.10% DMSO) | | | | | |
|---|---|---|---|---|---|---|---|
| | nM | hPPAR-α | hPPAR-δ | hPPAR-γ | rPPAR-α | rPPAR-δ | rPPAR-γ |
| 15-HEPE-EE | 137 | 0.90 | 1.4 | 1.0 | 1.1 | 1.3 | 1.2 |
| | 412 | 1.1 | 2.1* | 1.1 | 1.2 | 1.5 | 1.2 |
| | 1,235 | 1.1 | 2.1* | 1.3 | 1.2 | 1.6 | 1.4 |
| | 3,704 | 2.1* | 2.4* | 1.5 | 1.4 | 1.8 | 1.4 |
| | 11,111 | 4.2* | 5.6* | 2.1* | 1.7 | 2.4* | 1.8 |
| | 33,333 | 6.8* | 4.1* | 3.6* | 2.6* | 2.7* | 3.5* |
| | 100,000 | 5.0* | 8.0* | 6.1* | 1.4 | 7.2* | 10* |
| | 300,000 | 0.98 | 0.073 | 0.027 | 0.068 | 1.9 | 14* |
| EPA-EE | 137 | 0.88 | 1.2 | 0.90 | 1.3 | 1.3 | 0.90 |
| | 412 | 1.0 | 1.2 | 0.94 | 1.3 | 1.3 | 0.94 |
| | 1,235 | 0.90 | 1.3 | 0.94 | 1.3 | 1.2 | 0.99 |
| | 3,704 | 0.89 | 1.5 | 0.88 | 1.3 | 1.5 | 0.97 |
| | 11,111 | 0.86 | 1.4 | 1.0 | 1.4 | 1.4 | 0.97 |
| | 33,333 | 0.95 | 1.8 | 0.78 | 1.3 | 1.5 | 0.99 |
| | 100,000 | 1.1 | 2.6* | 1.2 | 1.3 | 2.0 | 1.0 |
| | 300,000 | 2.0* | 3.8* | 2.8* | 1.5 | 3.6* | 2.1* |

*Statistically significant (vs. vehicle).

### Example 2

### PPAR Antagonist and Live Cell Multiplex Activities

This example demonstrates that 15-HEPE ethyl ester exhibits apparent inhibition of human and rat PPARs at higher test concentrations, while EPA ethyl ester does not exhibit similar inhibitory activity.

### 2.1 Study Design

This study utilized proprietary reporter cells expressing a hybrid receptor comprising the N-terminal Gal4 DNA binding domain fused to the ligand binding domain of the specific human nuclear receptor (hPPARα, hPPARδ, and hPPARγ) and rat nuclear receptor (rPPARα, rPPARδ, and rPPARγ). The reporter vectors used in this Example comprise the firefly luciferase gene functionally linked to the Gal4 upstream activation sequence.

### 2.2 Methods

Nuclear Receptor assay was performed by suspending Reporter Cells in CRM containing 10% charcoal stripped FBS. 2×-EC80 concentrations of the appropriate reference agonist were then added to the Reporter Cell suspension, and 100 μL of the mixture dispensed into wells of a white 96-well plate. Test compounds were diluted using compound screening medium (CSM) containing 10% charcoal stripped FBS to generate “2×-concentration” treatment media. Immediately after this dilution step, 100 μL of each diluted 2×-concentration treatment medium was dispensed (in triplicate) into the Reporter Cell-containing assay wells.

After incubation at 37° C. for 24 hours, the treatment media was discarded. Each well was rinsed once with LCM Buffer; LCM substrate was then added. After incubation for 30 minutes at 37° C., fluorescence was measured to determine the relative number of live cells per assay well. LCM substrate was then discarded and 100 μL of Luciferase Detection Reagent substrate was added to each well. RLUs were quantified from each well to determine nuclear receptor activities.

### 2.3 Results

Live cell multiplex and antagonist assay results are shown in Table 2 below for 15-HEPE (ethyl ester, (S)-enantiomer). For comparison, agonist assay results for 15-HEPE's corresponding 15-lipoxygenase precursor, EPA ethyl ester, are also shown. Assay results for vehicle (DMSO) only were used to normalize the data (% inhibition=0%; % live cells=100%).

TABLE 2
Live Cell Multiplex and Antagonist Assay Results vs. Vehicle (0.10% DMSO)

| | | hPPAR-α | | hPPAR-δ | | hPPAR-γ | | rPPAR-α | | rPPAR-δ | | rPPAR-γ | |
|---|---|---|---|---|---|---|---|---|---|---|---|---|---|
| | nM | % In | % LC | % In | % LC | % In | % LC | % In | % LC | % In | % LC | % In | % LC |
| 15-HEPE-EE | 137 | 49 | 96 | 0.68 | 95 | 17 | 86 | 42 | 89 | 25 | 99 | 10 | 98 |
| | 412 | 21 | 94 | −7.7 | 98 | 0.87 | 99 | 26 | 98 | 1.5 | 97 | −13 | 101 |
| | 1,235 | 13 | 119 | −11 | 99 | −1.4 | 101 | 25 | 97 | −1.8 | 98 | −7.6 | 101 |
| | 3,704 | 14 | 120 | −10 | 100 | −4.6 | 101 | 21 | 99 | 6.8 | 99 | −6.7 | 100 |
| | 11,111 | 14 | 117 | −10 | 101 | −0.42 | 100 | 21 | 99 | 1.2 | 100 | −4.1 | 101 |
| | 33,333 | 26 | 115 | 1.6 | 101 | 5.2 | 99 | 38 | 98 | 26 | 100 | 8.4 | 101 |
| | 100,000 | 40 | 69 | 29 | 101 | 44 | 91 | 79 | 76 | 58 | 99 | 26 | 99 |
| | 300,000 | 83 | 35 | 100 | 7.7 | 100 | 4.9 | 100 | 3.1 | 100 | 5.0 | 93 | 91 |
| EPA-EE | 137 | 35 | 99 | −3.4 | 97 | 13 | 88 | 51 | 89 | 26 | 100 | 9.6 | 97 |
| | 412 | 20 | 93 | −8.9 | 97 | 3.8 | 99 | 20 | 98 | 4.0 | 94 | −7.4 | 101 |
| | 1,235 | 18 | 113 | −5.5 | 99 | −8.0 | 101 | 5.7 | 102 | 5.7 | 98 | −7.3 | 102 |
| | 3,704 | 17 | 112 | −15 | 100 | −11 | 101 | 4.2 | 103 | 8.1 | 95 | −0.55 | 101 |
| | 11,111 | 11 | 116 | −7.9 | 101 | −8.4 | 101 | −4.7 | 102 | 3.7 | 100 | 2.8 | 100 |
| | 33,333 | 10 | 112 | −18 | 101 | 2.9 | 102 | 1.4 | 100 | 7.2 | 99 | 5.0 | 101 |
| | 100,000 | 7.1 | 96 | −6.8 | 103 | −0.59 | 103 | 13 | 95 | 10 | 101 | 6.2 | 98 |
| | 300,000 | 2.8 | 80 | 11 | 103 | 30 | 92 | 36 | 94 | 36 | 102 | 7.0 | 102 |

% In = % Inhibition of agonist-stimulated receptor activity vs. 0.10% DMSO (=0%)
% LC = % Live cells vs. 0.10% DMSO (=100%)

1. A method for treating or preventing a disease mediated by peroxisome proliferator-activated receptors (PPARs) in a subject comprising, administering to the subject pharmaceutical composition comprising 15-HEPE to treat or prevent the disease in the subject.

2. The method of claim 1, wherein the disease is selected from impaired insulin sensitivity, psoriasis, cancer, fibrosis, melanoma, neurodegenerative disorders, Huntington's disease, inflammatory diseases, adipocyte differentiation, fertility or reproduction diseases, pain, and obesity.

3. The method of claim 1, wherein the 15-HEPE is present in the composition in an amount from about 50 mg to about 1000 mg.

4. The method of claim 1, wherein the 15-HEPE represents at least about 90% of all fatty acids present in the composition.

5. The method of claim 1, wherein the 15-HEPE represents substantially all fatty acids present in the composition.

6. The method of claim 1, wherein the 15-HEPE represents all fatty acids present in the composition.

7. The method of claim 1 wherein the composition is free of any other omega-3 fatty acids.

8. The method of claim 1, wherein the administering step includes administering the pharmaceutical composition to the human subject about 1 to about 4 times per day.

9. The method of claim 1, wherein the pharmaceutical composition comprises an orally deliverable capsule.

10. The method of claim 1, wherein the human subject has a predisposition to and/or a diagnosis of the liver disease.

11. A method for treating or preventing a disease mediated by peroxisome proliferator-activated receptors (PPARs) in a subject comprising, administering to the subject pharmaceutical composition comprising about 100 mg to about 5 g 15-HEPE to treat or prevent the disease in the subject.
